(19) United States
(12) Patent Application Publication
(10) Pub. No.: US 2017/0311956 A1
(43) Pub. Date: Nov. 2, 2017

(54) SURGICAL INSTRUMENT AND METHOD OF USE

(71) Applicant: Warsaw Orthopedic, Inc, Warsaw, IN (US)

(72) Inventors: William Alan Rezach, Covington, TN (US); Dustin Bobbitt, Hernando, MS (US)

(21) Appl. No.: 15/142,946

(22) Filed: Apr. 29, 2016

(51) Int. Cl. A61B 17/15; A61B 17/16; A61B 17/00

(52) U.S. Cl. CPC A61B 17/151; A61B 17/1671; A61B 2017/00725

(57) ABSTRACT

A surgical instrument comprises an arm. A first member is movable relative to the arm and gravity responsive to define a first orientation of the arm. A lock is engageable with the first member. A second member is movable relative to the arm and gravity responsive to measure an angle of a second orientation of the arm relative to the first orientation. Systems and methods of use are disclosed.

### TECHNICAL FIELD

The present disclosure generally relates to medical devices for the treatment of spinal disorders, and more particularly to a surgical instrument and method for treatment of a spine disorder.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. As part of these surgical treatments, implants, such as, for example, spinal constructs including plates, rods, fasteners and interbody devices are often employed for stabilization of a treated section of a spine. During such surgical treatments, surgical instruments may be used to facilitate delivery and placement of the implants. This disclosure describes an improvement over these prior technologies.

### SUMMARY

In one embodiment, a surgical instrument is provided. The surgical instrument comprises an arm. A first member is movable relative to the arm and gravity responsive to define a first orientation of the arm. A lock is engageable with the first member. A second member is movable relative to the arm and gravity responsive to measure an angle of a second orientation of the arm relative to the first orientation. In some embodiments, systems, spinal constructs, implants and methods are disclosed.

### BRIEF DESCRIPTION OF THE DRAWINGS

The present disclosure will become more readily apparent from the specific description accompanied by the following drawings, in which:

FIG. 1 is a perspective view of components of one embodiment of a surgical system in accordance with the principles of the present disclosure;

FIG. 2 is a side view of the components shown in FIG. 1;

FIG. 3 is a perspective view of the components shown in FIG. 1;

FIG. 4 is a side view of the components shown in FIG. 1;

FIG. 5 is an enlarged break away view of the components shown in FIG. 1;

FIG. 6 is a perspective view of the components shown in FIG. 1;

FIG. 7 is a side view of components of one embodiment of a surgical system in accordance with the principles of the present disclosure disposed with vertebrae;

FIG. 8 is a side view of components of one embodiment of a surgical system in accordance with the principles of the present disclosure disposed with vertebrae;

FIG. 9 is a side view of components of one embodiment of a surgical system in accordance with the principles of the present disclosure;

FIG. 10 is a perspective view of the components shown in FIG. 9;

FIG. 11 is an enlarged break away view of the components shown in FIG. 9;

FIG. 12 is an enlarged break away view of the components shown in FIG. 9;

FIG. 13 is an enlarged break away view of the components shown in FIG. 9;

FIG. 14 is a perspective view of components of one embodiment of a surgical system in accordance with the principles of the present disclosure; and

FIG. 15 is an enlarged break away view of the components shown in FIG. 14.

### DETAILED DESCRIPTION

The exemplary embodiments of the system and related methods of use disclosed are discussed in terms of medical devices for the treatment of musculoskeletal disorders and more particularly, in terms of a surgical system and method for treatment of a spine disorder. In some embodiments, the present surgical system comprises one or more surgical instruments that can be employed with a spinal construct for treating a spine disorder. In some embodiments, the present surgical system can be employed with a correction treatment, for example, a pedicle subtraction osteotomy (PSO) to correct angular and fixed kyphotic deformity, such as post traumatic deformity, congenital deformity and/or post infectious deformity.

In some embodiments, the present surgical system comprises a surgical instrument, such as, for example, an angle gauge. In some embodiments, the angle gauge includes a dual pendulum angle measurement device. In some embodiments, the angle gauge is configured to facilitate measuring angle cuts during a PSO procedure. In some embodiments, the angle gauge is configured to provide an angular relationship between the surgical instrument and an implant and/or anatomy. In some embodiments, the angle gauge is configured to utilize gravity assisted measurement.

In some embodiments, the present surgical system includes a surgical instrument employed with a method that includes the step of disposing a first pendulum of the surgical instrument on a reference point. In some embodiments, the method includes the step of locking the first pendulum for calibration. In some embodiments, the method includes the step of moving a second pendulum of the surgical instrument relative to the first pendulum to provide an angle measurement. In some embodiments, the surgical instrument includes a calibration button.

In some embodiments, the surgical instrument includes a relative angle indicator. In some embodiments, the angle indicator includes indicia comprising a needle indicator. In some embodiments, the surgical instrument measures a change in an angular position of the surgical instrument relative to a vertical plane using a small sphere inside of a pendulum scale. In some embodiments, the surgical instrument includes a mechanical, cleanable, reusable, gravity based angle measuring device to aid in the carpentry of orthopedic/spinal surgery. In some embodiments, the surgical instrument is configured to allow a practitioner to more accurately make cuts, drill holes or implant devices relative to other anatomical structures.

In some embodiments, the surgical instrument comprises an angle gauge that includes a shaft. In some embodiments, the angle gauge includes a locking screw. In some embodiments, the angle gauge includes a scale. In some embodiments, the angle gauge includes a spherical ball bearing disposed within the scale. In some embodiments, disengagement of the locking screw allows the scale to act as a pendulum. In some embodiments, the scale is configured to freely pivot to orient a zero point of the scale to fall directly below the pivot point. In some embodiments, engaging the locking screw with the scale fixes the orientation of the scale relative to the shaft. In some embodiments, the ball bearing is configured to roll within the scale as the surgical instrument is moved to indicate a relative angle change within the scale.

In some embodiments, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the present disclosure may be employed with other osteal and bone related applications, including those associated with diagnostics and therapeutics. In some embodiments, the disclosed surgical system may be alternatively employed in a surgical treatment with a patient in a prone or supine position, and/or employ various surgical approaches to the spine, including anterior, posterior, posterior mid-line, direct lateral, postero-lateral, and/or antero lateral approaches, and in other body regions. The present disclosure may also be alternatively employed with procedures for treating the lumbar, cervical, thoracic, sacral and pelvic regions of a spinal column. The surgical system of the present disclosure may also be used on animals, bone models and other non-living substrates, such as, for example, in training, testing and demonstration.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. In some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

As used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. In some embodiments, as used in the specification and including the appended claims, the term “tissue” includes soft tissue, ligaments, tendons, cartilage and/or bone unless specifically referred to otherwise.

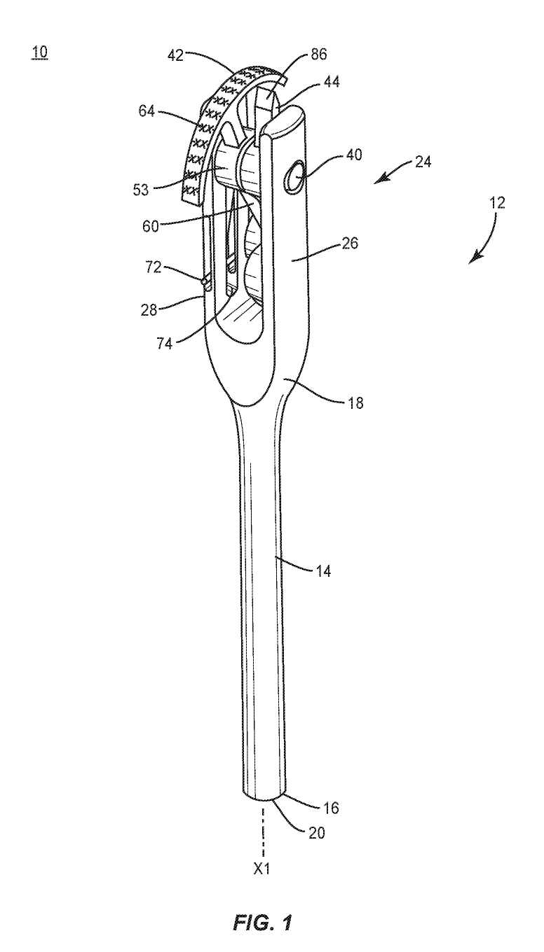

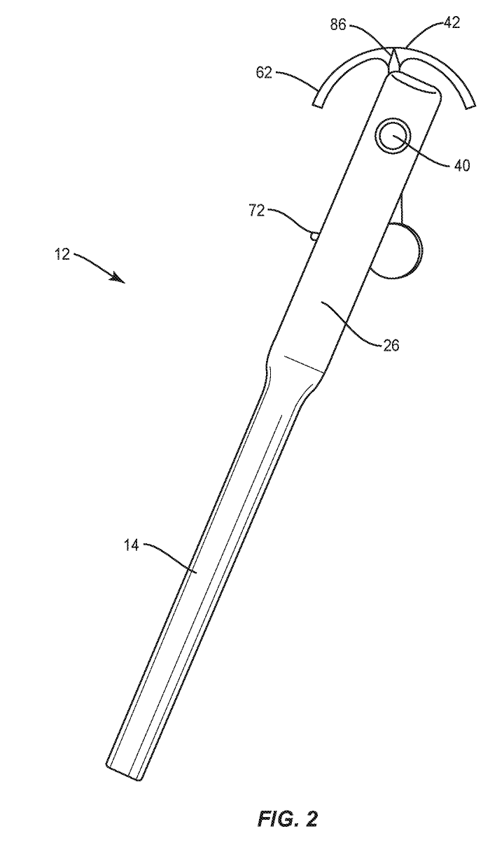

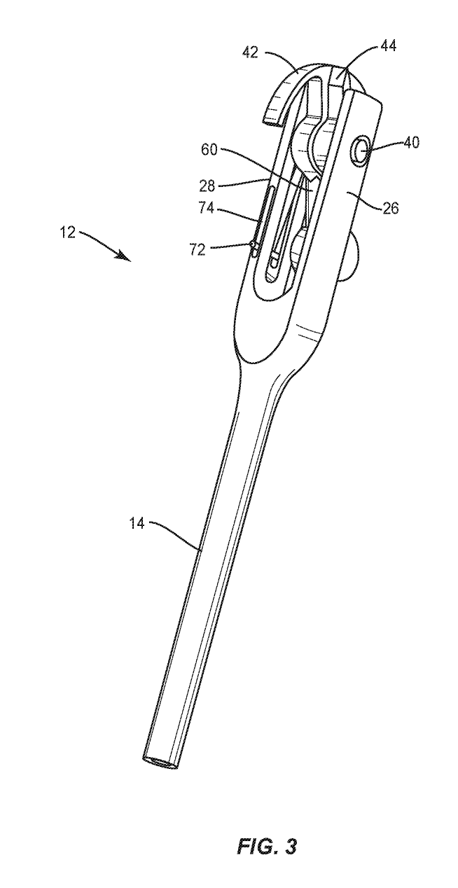

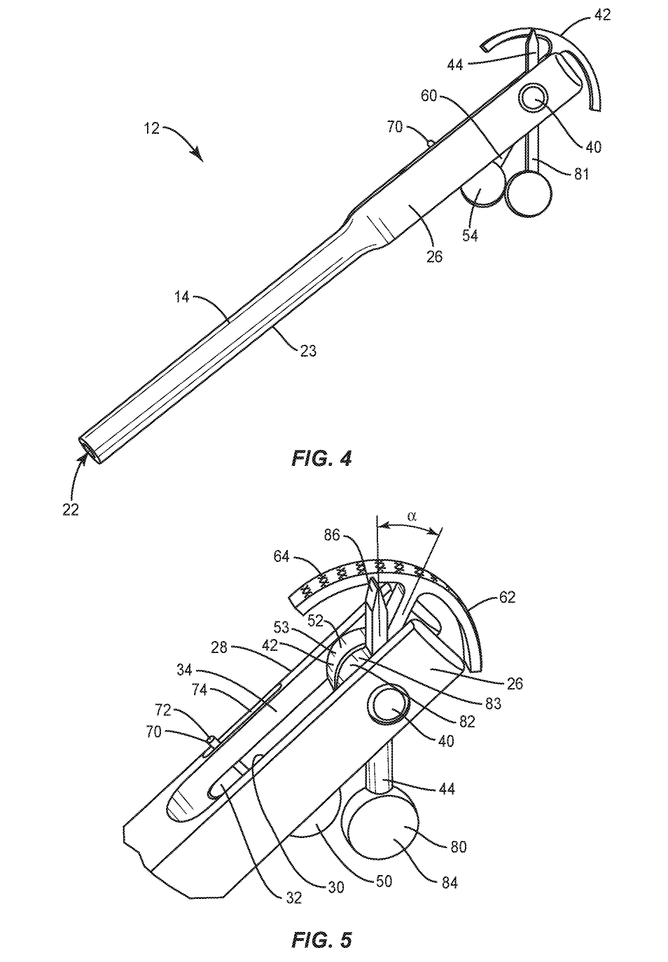

The following discussion includes a description of a surgical system including a surgical instrument, related components and methods of employing the surgical system in accordance with the principles of the present disclosure. Alternate embodiments are disclosed. Reference is made in detail to the exemplary embodiments of the present disclosure, which are illustrated in the accompanying figures. Turning to FIGS. 1-6, there are illustrated components of a surgical system 10 including a surgical instrument, as described herein.

The components of surgical system 10 can be fabricated from biologically acceptable materials suitable for medical applications, including metals, synthetic polymers, ceramics and bone material and/or their composites. For example, the components of surgical system 10, individually or collectively, can be fabricated from materials such as stainless steel alloys, commercially pure titanium, titanium alloys, Grade 5 titanium, super-elastic titanium alloys, cobalt-chrome alloys, superelastic metallic alloys (e.g., Nitinol, super last-plastic metals, such as GUM METAL®), ceramics and composites thereof such as calcium phosphate (e.g., SKELITE™), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations.

Various components of surgical system 10 may have material composites, including the above materials, to achieve various desired characteristics such as strength, rigidity, elasticity, compliance, biomechanical performance, durability and radiolucency or imaging preference. The components of surgical system 10, individually or collectively, may also be fabricated from a heterogeneous material such as a combination of two or more of the above-described materials. The components of surgical system 10 may be monolithically formed, integrally connected or include fastening elements and/or instruments, as described herein.

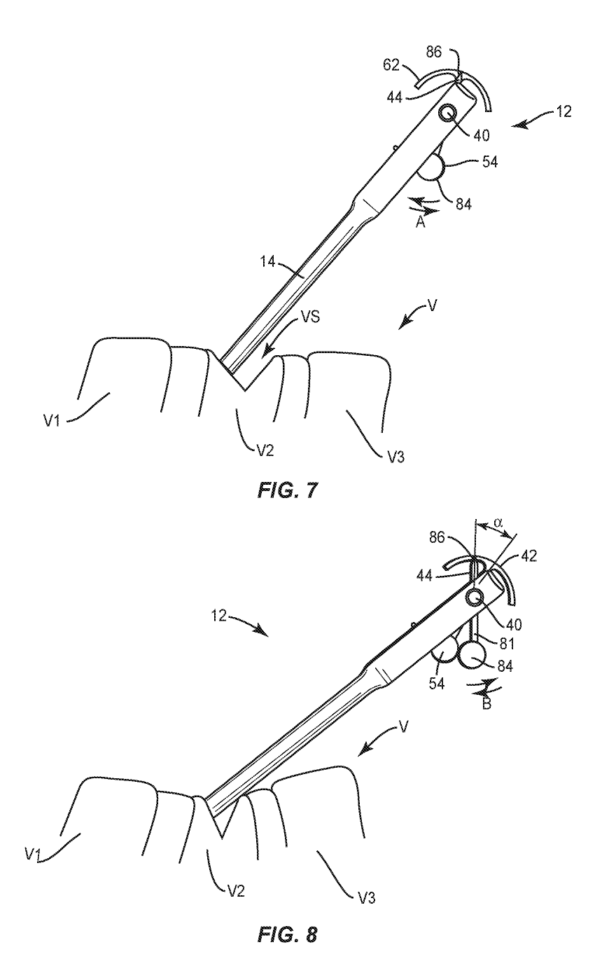

Surgical system 10 includes a surgical instrument, such as, for example, an angle gauge 12. Gauge 12 is gravity responsive to measure a change in angle between a first orientation, for example, as shown in FIG. 7, and a second orientation, for example, as shown in FIG. 8, as described herein. Gauge 12 includes an arm 14 that extends between an end 16 and an end 18 defining a longitudinal axis X1. In some embodiments, arm 14 may have various cross-sectional geometries, such as, for example oval, oblong, triangular, square, polygonal, irregular, uniform, non-uniform, offset, staggered, undulating, arcuate, variable and/or tapered. In some embodiments, arm 14 includes an outer gripping surface 23 configured to facilitate gripping by a practitioner. Gripping surface 23 may be, for example, rough, arcuate, undulating, mesh, porous, semi-porous, dimpled and/or textured.

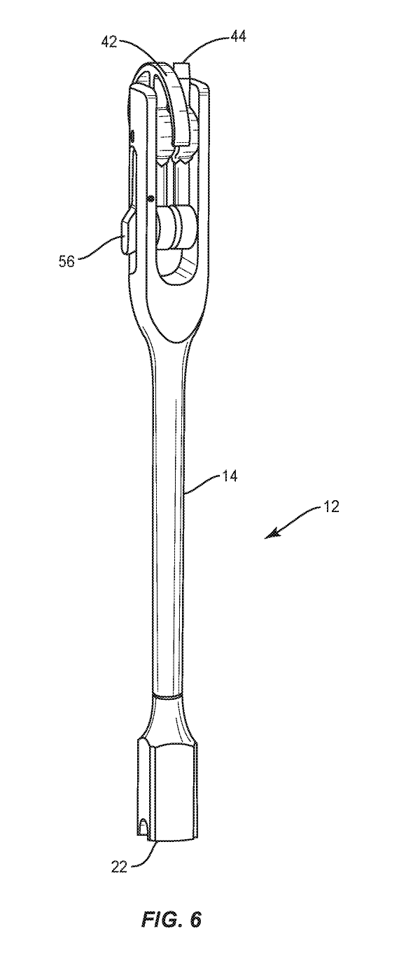

End 16 includes an engagement surface 20 configured for connection with a spinal implant or an anatomy, as described herein. In some embodiments, surface 20 defines a cavity 22, as shown in FIG. 6, configured for disposal of a spinal implant and/or an anatomy. In some embodiments, cavity 22 includes walls configured to engage a spinal implant. In some embodiments, the walls include a mating element configured to facilitate engagement with a spinal implant. In some embodiments, cavity 22 includes a counter torque connection configured for engagement with a spinal implant to measure a distraction and/or compression angle of vertebrae. In some embodiments, all or only a portion of cavity 22 may have cross section configurations, such as, for example, oval, oblong triangular, square, polygonal, irregular, uniform, non-uniform, offset, staggered, and/or tapered. In some embodiments, the spinal implant can include a spinal rod, bone fastener, connector, plate and/or interbody device. In some embodiments, the anatomy includes tissue, as described herein.

End 18 includes a bifurcated portion 24 having an extension 26 and an extension 28. Extension 26 includes a surface 30 that defines a portion of a cavity 32 configured for disposal of gauge members, as described herein. Extension 28 includes a surface 34 that defines a portion of cavity 32. In some embodiments, cavity 32 is U-shaped. In some embodiments, cavity 32 may have various cross-sectional geometries, such as, for example oval, oblong, triangular, square, polygonal, irregular, uniform, non-uniform, offset, staggered, undulating, arcuate, variable and/or tapered. Extension 26 is connected with extension 28 by a pin 40. Pin 40 extends between extensions 26, 28 transverse to axis X1.

Gauge 12 includes a member, such as, for example, a pendulum 42 that extends between an end 50 and an end 52. End 52 is connected with end 50 via an extension 60. End 50 includes a weight 54 that rotates pendulum 42 relative to arm 14, as described herein. End 52 includes a circumferential band 53, which is disposed about a pivot or pin 40 and configured to facilitate rotation of pendulum 42 relative to arm 14. In some embodiments, band 53 is disposed with pin 40 in a substantially frictionless engagement to facilitate rotation of pendulum 42 relative to arm 14. In some embodiments, the surface of band 53 and/or pin 40 can be smooth, even, rough, textured, porous, semi-porous, dimpled and/or polished for engagement therebetween to facilitate rotation of pendulum 42 relative to arm 14.

In some embodiments, pin 40 is fixed with pendulum 42 and rotatable within the inner surfaces of extensions 26, 28 such that pendulum 42 rotates relative to arm 14. In some embodiments, pin 40 is fixed with pendulum 42 and connected with the inner surfaces of extensions 26, 28 in a substantially frictionless engagement to facilitate rotation of pendulum 42 relative to arm 14. In some embodiments, the surface of pin 40 and/or the inner surfaces of extensions 26, 28 can be smooth, even, rough, textured, porous, semi-porous, dimpled and/or polished for engagement therebetween to facilitate rotation of pendulum 42 relative to arm 14.

Weight 54 is suspended from pin 40 via band 53 in a gravity responsive configuration such that weight 54 can rotate freely about pin 40, as described herein. In some embodiments, weight 54 is displaced and/or rotated from a resting and/or equilibrium orientation of pendulum 42 and/or gauge 12 and a restoring force due to gravity is subjected to weight 54 to accelerate and rotate weight 54 back toward a resting and/or equilibrium orientation of pendulum 42. In some embodiments, pendulum 42 is disposed in a non-locking orientation, as described herein, such that the restoring force combined with weight 54 causes pendulum 42 to oscillate and/or rotate about a resting and/or equilibrium orientation in a first direction and/or a second, opposing direction.

End 52 includes an arcuate portion 62 extending therefrom that includes indicia 64. In some embodiments, indicia 64 includes information representing and displaying an angular measurement, as described herein. In some embodiments, indicia 64 includes graduated markings disposed along a surface of portion 62. In some embodiments, the markings display, represent and/or provide information relating to an angular range for measuring, selecting, adjusting and/or displaying an angle measured by gauge 12, as described herein. In some embodiments, the markings may include bi-laterally disposed grooves equidistantly spaced apart and corresponding to measured angular increments of indicia 64.

In some embodiments, indicia 64 includes markings that may be disposed in increments of 10 angular degrees. In some embodiments, indicia 64 may include an analog, such as, for example, a dial with a numerical indicator of angle and/or digital display, such as, for example, LED and/or LCD. In some embodiments, indicia 64 include human readable visual indicia, such as, for example, a label, color coding, alphanumeric characters or an icon. In some embodiments, indicia 64 include human readable tactile indicia, such as, for example, raised portions, lowered portions or Braille. In some embodiments, indicia 64 is a printed or written item in combination with a slot or groove, whereby the printed or written item is placed in the slot or groove to display information. In some embodiments, indicia 64 may be applied as an adhesive.

Arm 14 includes a lock 70, which is selectively engageable to fix pendulum 42 relative to arm 14, for example, in a resting, zero angle, calibration and/or equilibrium orientation. In some embodiments, lock 70 includes a locking pin 72, as shown in FIG. 5. In some embodiments, pin 72 is translatable within a cavity 74 of extension 26 to engage and fix pendulum 42 relative to arm 14. In some embodiments, lock 70 is actuated to fix pendulum 42 relative to arm 14 in first orientation, such as, for example, a zero angle or calibration orientation, as shown in FIG. 7 and described herein.

Gauge 12 includes a member, such as, for example, a pendulum 44 that extends between an end 80 and an end 82. End 82 is connected with end 80 via an extension 81. End 80 includes a weight 84 that rotates pendulum 44 relative to arm 14, as described herein. End 82 includes a circumferential band 83, which is disposed about a pivot or pin 40 and configured to facilitate rotation of pendulum 44 about pin 40 relative to arm 14. In some embodiments, band 83 is disposed with pin 40 in a substantially frictionless engagement to facilitate rotation of pendulum 44 relative to arm 14. In some embodiments, the surface of band 83 and/or pin 40 can be smooth, even, rough, textured, porous, semi-porous, dimpled and/or polished for engagement therebetween to facilitate rotation of pendulum 44 relative to arm 14.

In some embodiments, pin 40 is fixed with pendulum 44 and rotatable within the inner surfaces of extensions 26, 28 such that pendulum 44 rotates relative to arm 14. In some embodiments, pin 40 is fixed with pendulum 44 and connected with the inner surfaces of extensions 26, 28 in a substantially frictionless engagement to facilitate rotation of pendulum 44 relative to arm 14. In some embodiments, the surface of pin 40 and/or the inner surfaces of extensions 26, 28 can be smooth, even, rough, textured, porous, semi-porous, dimpled and/or polished for engagement therebetween to facilitate rotation of pendulum 44 relative to arm 14.

Weight 84 is suspended from pin 40 via band 83 in a gravity responsive configuration such that weight 84 can rotate freely about pin 40, as described herein. In some embodiments, weight 84 is displaced and/or rotated from a resting, zero angle, calibration and/or equilibrium orientation of pendulum 44 and/or gauge 12 and a restoring force due to gravity is subjected to weight 84 to accelerate and rotate weight 84 back toward a resting and/or equilibrium orientation of pendulum 44. In some embodiments, pendulum 44 is disposed in a non-locking orientation, as described herein, such that the restoring force combined with weight 84 causes pendulum 44 to oscillate and/or rotate about a resting and/or equilibrium orientation in a first direction and/or a second, opposing direction.

End 82 includes a pointer 86 extending therefrom. Pointer 86 is disposable adjacent surface 62 for alignment with indicia 64 to represent and display an angular measurement, as described herein. For example, pointer 86 is configured to indicate angle α relative to pendulum 42, for example, as shown in FIG. 8 and described herein.

Pin 40 connects pendulum 42 and pendulum 44 to arm 14. In some embodiments, pendulum 42 and/or pendulum 44 rotate about pin 40 relative to arm 14 to measure an angle, a zero or calibration angle, a relative angle, an angular orientation and/or a change in an angular orientation, such as, for example, an angle α, of gauge 12, a spinal implant or an anatomy measured by and/or connected with gauge 12, as described herein.

For example, gauge 12 is disposable in a first orientation, such as, for example, a calibration orientation, as shown in FIG. 7. Lock 70 is disengaged from pendulum 42 such that pendulum 42 is disposed in a non-locking orientation and weight 54 rotates pendulum 42 about a resting and/or equilibrium orientation. Gauge 12 is manipulated to a calibration orientation such that surface 20 engages a spinal implant and/or anatomy and is disposed in a selected orientation with the spinal implant and/or anatomy. Weight 54 rotates from rest and/or equilibrium and a restoring force due to gravity is subjected to weight 54. As such, pendulum 42 is rotated, in the directions shown by arrows A in FIG. 7, to a calibration orientation. Lock 70 engages pendulum 42 in a locked orientation to fix pendulum 42 relative to arm 14 in the calibration orientation.

Gauge 12 is disposable in a second orientation, such as, for example, an angle measurement orientation, as shown in FIG. 8, relative to the calibration orientation. Pendulum 42 is disposed in the locked orientation with arm 14, and weight 84 rotates pendulum 44 about a resting and/or equilibrium orientation. Gauge 12 is manipulated such that surface 20 engages the same or a different spinal implant and/or the same or a different portion of the anatomy for disposal in a selected orientation relative to the position of gauge 12 in the calibration orientation. Weight 84 rotates from rest and/or equilibrium and a restoring force due to gravity is subjected to weight 84. As such, pendulum 44 is rotated, in the directions shown by arrows B in FIG. 8, to an angle measurement orientation relative to the calibration orientation of pendulum 42. Pendulum 44 rotates relative to pendulum 42 such that pointer 86 is aligned with indicia 64 to represent and display an angular measurement of the angular difference between the engagement of surface 20 with the spinal implant and/or anatomy of gauge 12 in the calibration orientation and the engagement of surface 20 with the spinal implant and/or anatomy of gauge 12 in the angular measurement orientation, for example, angle α shown in FIG. 8.

In assembly, operation and use, surgical system 10 including gauge 12, similar to the systems and methods described with regard to FIGS. 1-6, is employed with a surgical procedure, such as, for example, a PSO for treatment of a spine of a patient including vertebrae V, as shown in FIGS. 7 and 8. Surgical system 10 may also be employed with other surgical procedures, such as, for example, discectomy, laminectomy, fusion, laminotomy, laminectomy, nerve root retraction, foramenotomy, facetectomy, decompression, spinal nucleus or disc replacement and bone graft and implantable prosthetics including plates, rods, and bone engaging fasteners.

Surgical system 10 is employed with a PSO procedure for treatment of an applicable condition or injury of an affected section of a spinal column and adjacent areas within a body. For example, vertebrae V includes a vertebral level V1, a vertebral level V2 and a vertebral level V3. Diseased and/or damaged vertebrae and intervertebral discs are disposed at vertebrae V2 between vertebrae V1 and V3.

In use, to treat the affected section of vertebrae V, a medical practitioner obtains access to a surgical site including vertebrae V, in any appropriate manner, such as through incision and retraction of tissues. In some embodiments, surgical system 10 may be used in any existing surgical method or technique including open surgery, mini-open surgery, minimally invasive surgery and percutaneous surgical implantation, whereby vertebrae V is accessed through a mini-incision, or sleeve that provides a protected passageway to the area.

An incision is made in the body of a patient and a cutting instrument (not shown) creates a surgical pathway for implantation of components of surgical system 10. A preparation instrument (not shown) can be employed to prepare tissue surfaces of vertebrae V, as well as for aspiration and irrigation of a surgical region.

In some embodiments, a surgical instrument, such as, for example, an osteotome (not shown) is utilized to facilitate removing all or a portion of vertebra V2 and adjacent intervertebral disc tissue to define a vertebral space VS, as shown in FIG. 7, to dispose cut surfaces of vertebra V2 at a first angular orientation. In some embodiments, a surgical instrument, such as, for example, a compressor/distractor (not shown) pivots vertebrae V about vertebral space VS to dispose the cut surfaces of vertebra V2 at a second angular orientation, as shown in FIG. 8, during a surgical correction treatment to rotate, displace, pull, twist or align vertebrae V to a selected orientation for sagittal, coronal and/or axial correction.

In connection with the surgical treatment, gauge 12 is employed to measure the relative angular orientation of the cut surfaces of vertebra V2, one or more spinal implants and/or other anatomy. Gauge 12 is disposed in a zero angle or calibration orientation, as shown in FIG. 7. Pendulum 42 is disposed in a non-locking orientation, as described herein. Surface 20 engages a cut surface of vertebra V2, as shown in FIG. 7, and gauge 12 is manipulated to a calibration orientation. Pendulum 42 is rotated, in the directions shown by arrows A in FIG. 7, to the calibration orientation, as described herein. Lock 70 engages pendulum 42 in a locked orientation to fix pendulum 42 relative to arm 14 in the calibration orientation.

Surface 20 engages a cut surface of vertebra V2, as shown in FIG. 8, and gauge 12 is manipulated to a measurement orientation relative to the position of gauge 12 in the calibration orientation. Pendulum 44 is rotated, in the directions shown by arrows B in FIG. 8, to an angle measurement orientation relative to the calibration orientation of pendulum 42. Pendulum 44 rotates relative to pendulum 42 such that pointer 86 is aligned with indicia 64 to display angle α shown in FIG. 8, which represents an angular measurement of the angular difference between the engagement of surface 20 with the cut surface of vertebra V2 shown in FIG. 7 in the calibration orientation and the engagement of surface 20 with the cut surface of vertebra V2 shown in FIG. 8 in the measurement orientation. In some embodiments, angle α includes one or a plurality of angular orientations in a range of 0 through 180 degrees. In some embodiments, angle α is measured to determine the size of a spinal implant, such as, for example, an intrabody implant.

Upon completion of a procedure, as described herein, the surgical instruments, assemblies and non-implanted components of surgical system 10 are removed and the incision(s) are closed. One or more of the components of surgical system 10 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of surgical system 10.

In some embodiments, surgical system 10 comprises a kit including a plurality of interbody devices, plates, bone fasteners and/or fixation elements, which may be employed with a single vertebral level or a plurality of vertebral levels. In some embodiments, the fasteners may be engaged with vertebrae in various orientations, such as, for example, series, parallel, offset, staggered and/or alternate vertebral levels. In some embodiments, the fasteners may be configured as multi-axial screws, sagittal angulation screws, pedicle screws, mono-axial screws, uni-planar screws, fixed screws, anchors, tissue penetrating screws, conventional screws and expanding screws. In some embodiments, the fasteners may be employed with wedges, anchors, buttons, clips, snaps, friction fittings, compressive fittings, expanding rivets, staples, nails, adhesives, posts, connectors, fixation plates and/or posts. In some embodiments, surgical system 10 includes surgical instruments, such as, for example, inserters, extenders, reducers, spreaders, distractors, blades, retractors, clamps, forceps, elevators and drills, which may be alternately sized and dimensioned, and arranged as a kit.

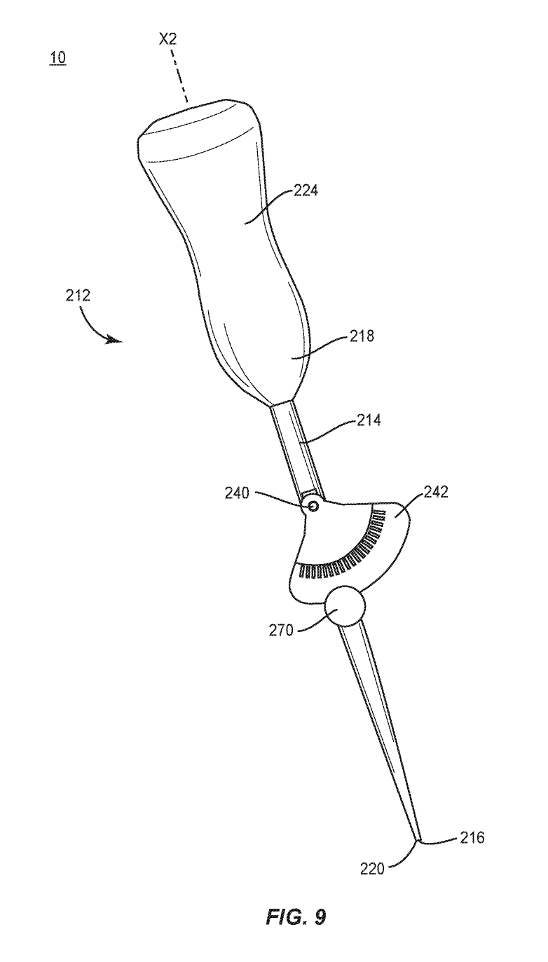

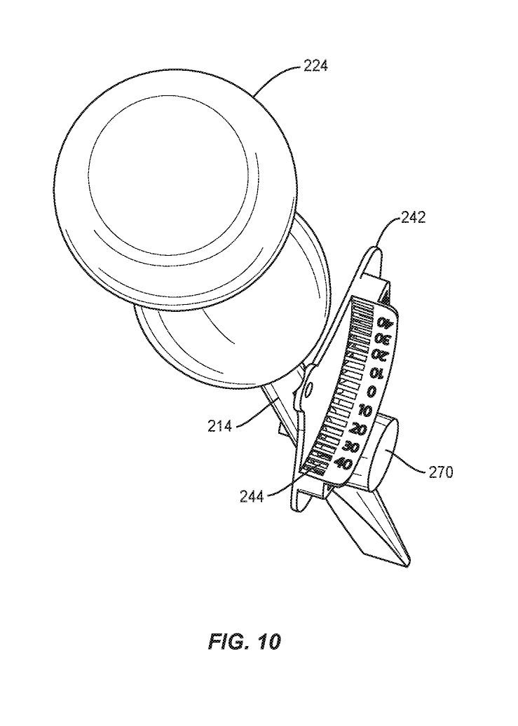

In one embodiment, as shown in FIGS. 7-13, surgical system 10, similar to the systems and methods described herein, includes a surgical instrument, such as, for example, an angle gauge 212, similar to gauge 12 described herein. Gauge 212 is gravity responsive to measure a change in angle between a first orientation and a second orientation, similar to that described herein. Gauge 212 includes an arm 214 that extends between an end 216 and an end 218, and defines a longitudinal axis X2. End 216 includes an engagement surface 220 configured for connection with a spinal implant or an anatomy, as described herein. End 218 includes a pin 240 extending transverse to axis X2.

Gauge 212 includes a member, such as, for example, a scale 242 that extends between an end 250 and an end 252. Scale 242 rotates relative to arm 214, as described herein. End 250 includes a ring 253, which is disposed about a pivot or pin 240 and configured to facilitate rotation of scale 242 relative to arm 214. In some embodiments, ring 253 is disposed with pin 240 in a substantially frictionless engagement to facilitate rotation of scale 242 relative to arm 214. In some embodiments, pin 240 is fixed with scale 242 and rotatable relative to arm 214 such that scale 242 rotates relative to arm 214. Scale 242 is suspended from pin 240 via ring 253 in a gravity responsive configuration such that scale 242 can rotate freely about pin 240, similar to that described herein. In some embodiments, scale 242 is displaced and/or rotated from a resting and/or equilibrium orientation of scale 242 and/or gauge 212 and a restoring force due to gravity is subjected to scale 242 to accelerate and rotate scale 242 back toward a resting and/or equilibrium orientation of scale 242. In some embodiments, scale 242 is disposed in a non-locking orientation, as described herein, such that the restoring force causes scale 242 to oscillate and/or rotate about a resting and/or equilibrium orientation in a first direction and/or a second, opposing direction.

End 252 includes an arcuate portion 262 that includes indicia 264, similar to indicia 64 described herein. Scale 242 includes a surface 280 that defines a track 282. Track 282 includes openings 284. In some embodiments, openings are aligned corresponding to indicia 264. Openings 284 are configured to display a location of a member, such as, for example, a ball 244, as described herein. Track 282 is configured for moveable disposal of ball 244 within track 282 for alignment with indicia 264 to display and represent an angular measurement, similar to that described herein.

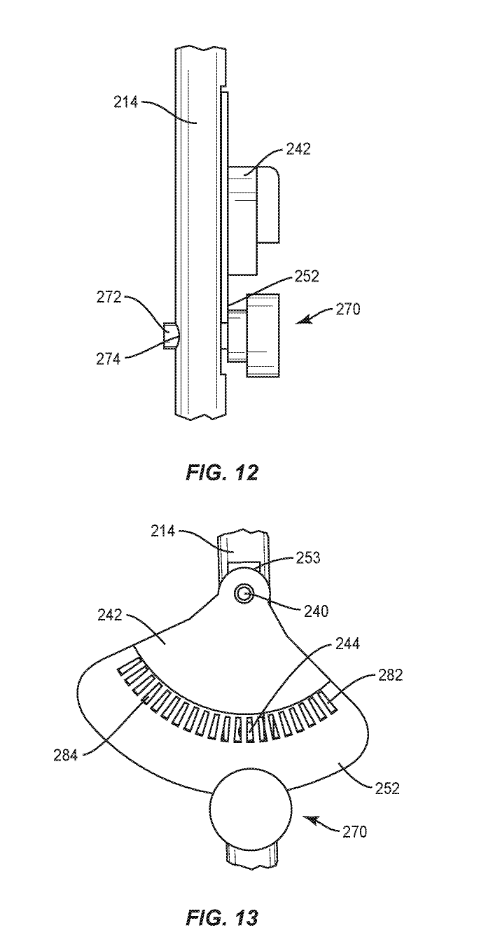

Arm 214 includes a lock 270, which is selectively engageable to fix scale 242 relative to arm 214, for example, in a resting, zero angle, calibration and/or equilibrium orientation. In some embodiments, lock 270 includes a locking pin 272, as shown in FIG. 12. In some embodiments, pin 272 is moveable within a cavity 274 of arm 214 to engage and fix scale 242 relative to arm 214. In some embodiments, lock 270 is actuated to fix scale 242 relative to arm 214 in first orientation, such as, for example, a zero angle or calibration orientation, similar to that described herein.

Gauge 212 includes ball 244 disposed within track 282. Ball 244 is configured to translate or roll along track 282 relative to arm 214 to display and represent an angular measurement. In some embodiments, the surface of track 282 can be smooth, even, rough, textured, porous, semi-porous, dimpled and/or polished for engagement therebetween to facilitate rotation of ball 244 relative to arm 214.

Ball 244 is disposed within track 282 and suspended with scale 242 from pin 240 in a gravity responsive configuration such that ball 244 can rotate freely within track 282 relative to scale 242 and/or arm 214, as described herein. In some embodiments, ball 244 is displaced and/or rotated from a resting, zero angle, calibration and/or equilibrium orientation of ball 244 and/or gauge 212 and a restoring force due to gravity is subjected to ball 244 to accelerate and rotate ball 244 back toward a resting and/or equilibrium orientation of ball 244. In some embodiments, the restoring force causes ball 244 to oscillate and/or rotate about a resting and/or equilibrium orientation in a first direction and/or a second, opposing direction relative to scale 242 and/or arm 214.

Pin 240 connects scale 242 to arm 214. In some embodiments, scale 242 and/or ball 244 rotates about pin 240 relative to arm 214 to measure an angle, a zero or calibration angle, a relative angle, an angular orientation and/or a change in an angular orientation, of gauge 212, a spinal implant or an anatomy measured by and/or connected with gauge 212, as described herein.

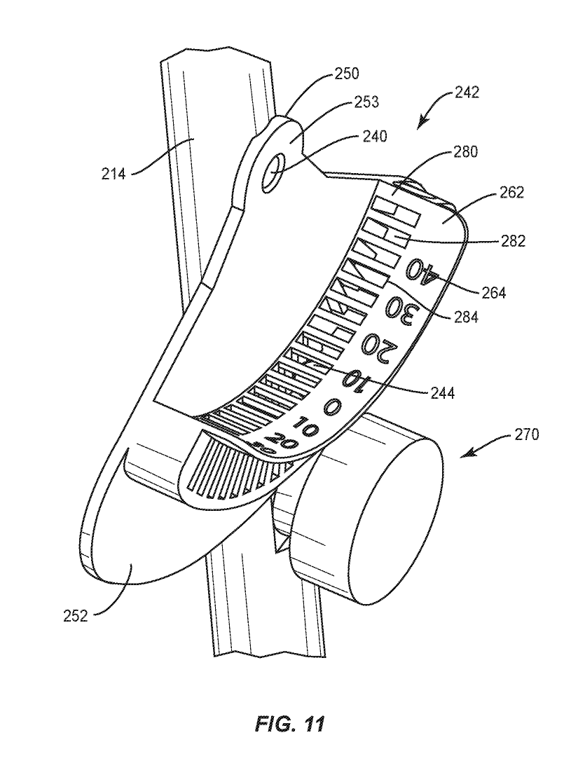

For example, gauge 212 is disposable in a first orientation, such as, for example, a calibration orientation, as shown in FIG. 11. Lock 270 is disengaged from scale 242 such that scale 242 is disposed in a non-locking orientation and scale 242 rotates about a resting and/or equilibrium orientation. Gauge 212 is manipulated to a calibration orientation such that surface 220 engages a spinal implant and/or anatomy and is disposed in a selected orientation with the spinal implant and/or anatomy. Scale 242 rotates from rest and/or equilibrium and a restoring force due to gravity is subjected to scale 242. As such, scale 242 is rotated to a calibration orientation, as described herein. Lock 270 engages scale 242 in a locked orientation to fix scale 242 relative to arm 214 in the calibration orientation.

Gauge 212 is disposable in a second orientation, such as, for example, an angle measurement orientation, as shown in FIG. 13, relative to the calibration orientation. Scale 242 is disposed in the locked orientation with arm 214, and ball 244 rotates about a resting and/or equilibrium orientation. Gauge 212 is manipulated such that surface 220 engages the same or a different spinal implant and/or the same or a different portion of the anatomy for disposal in a selected orientation relative to the position of gauge 212 in the calibration orientation. Ball 244 rotates from rest and/or equilibrium relative to scale 242 and/or arm 214 and a restoring force due to gravity is subjected to ball 244. As such, ball 244 rotates to an angle measurement orientation relative to the calibration orientation of scale 242. Ball 244 rotates relative to scale 242 such that ball 244 is aligned with indicia 264 to represent and display an angular measurement of the angular difference between the engagement of surface 220 with the spinal implant and/or anatomy of gauge 212 in the calibration orientation and the engagement of surface 220 with the spinal implant and/or anatomy of gauge 212 in the angular measurement orientation, similar to that described herein.

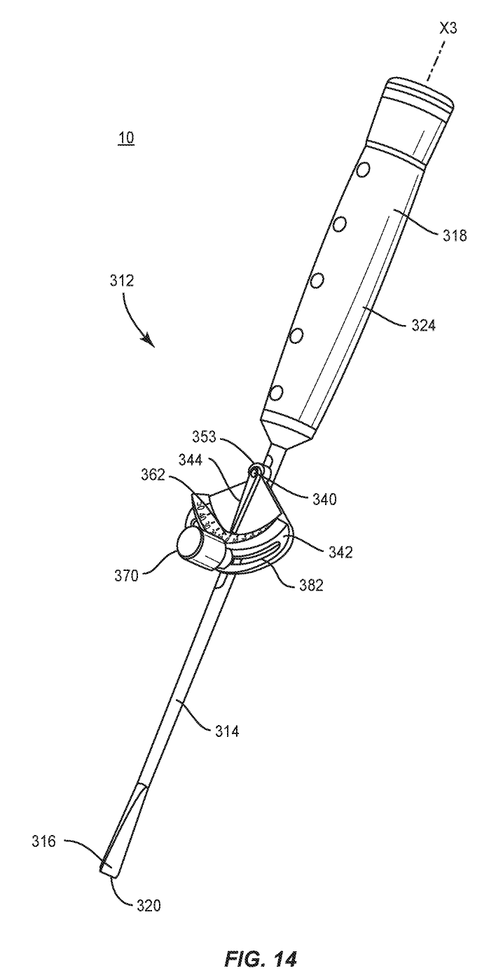

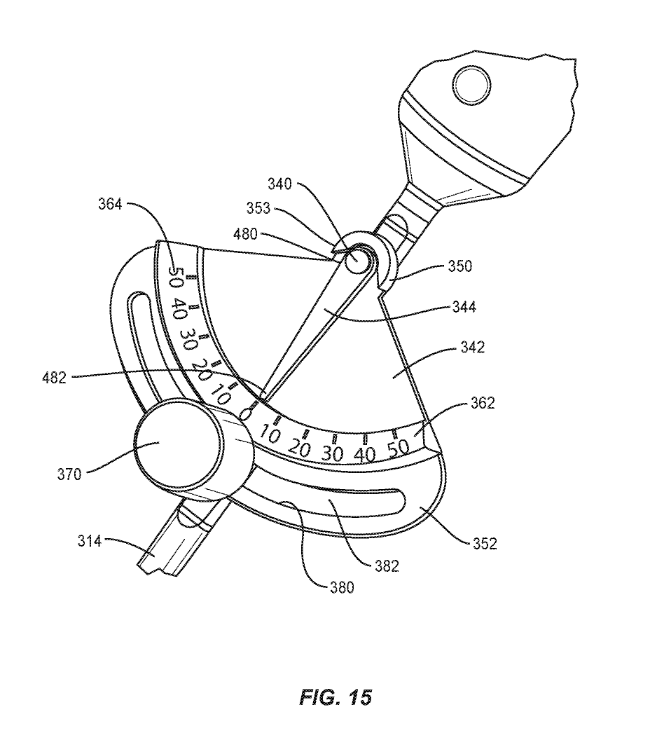

In one embodiment, as shown in FIGS. 14 and 15, surgical system 10, similar to the systems and methods described herein, includes a surgical instrument, such as, for example, an angle gauge 312, similar to gauge 212 described herein. Gauge 312 is gravity responsive to measure a change in angle between a first orientation and a second orientation, similar to that described herein. Gauge 312 includes an arm 314 that extends between an end 316 and an end 318, and defines a longitudinal axis X3. End 316 includes an engagement surface 320 configured for connection with a spinal implant or an anatomy, as described herein. End 318 includes a pin 340 extending transverse to axis X3, similar to that described herein.

Gauge 312 includes a scale 342, similar to scale 242. Scale 342 extends between an end 350 and an end 352. Scale 342 rotates relative to arm 314. End 352 includes a ring 353, which is disposed about a pivot or pin 340 and configured to facilitate rotation of scale 342 relative to arm 314. Scale 342 is suspended from pin 340 via ring 353 in a gravity responsive configuration, similar to that described herein, such that scale 342 can rotate freely about pin 340.

End 352 includes an arcuate portion 362 having indicia 364, similar to indicia 264 described herein. Scale 342 includes a surface 380 that defines a track 382. Track 382 is configured for moveable disposal of a lock 370, similar to lock 270 described herein. Arm 314 includes a lock 370, which is selectively engageable with track 382 to fix scale 342 relative to arm 314, for example, in a resting, zero angle, calibration and/or equilibrium orientation. In some embodiments, lock 370 is actuated to fix scale 342 relative to arm 314 in first orientation, such as, for example, a zero angle or calibration orientation, similar to that described herein.

Gauge 12 includes a member, such as, for example, a pointer 344 that extends between an end 380 and an end 382. Pointer 344 rotates relative to arm 14. End 380 is disposed about a pivot or pin 340 for rotation of pointer 344 about pin 340 relative to arm 314, similar to that described herein. Pointer 344 is suspended from pin 340 in a gravity responsive configuration, similar to that described herein, such that pointer 344 can rotate freely about pin 340. In some embodiments, pointer 344 is displaced and/or rotated from a resting, zero angle, calibration and/or equilibrium orientation of pointer 344 and/or gauge 312 and a restoring force due to gravity is subjected to pointer 344 to accelerate and rotate pointer 344 back toward a resting and/or equilibrium orientation of pointer 344.

Pin 340 connects scale 342 and pointer 344 to arm 314. In some embodiments, scale 342 and/or pointer 344 rotate about pin 340 relative to arm 314 to measure an angle, a zero or calibration angle, a relative angle, an angular orientation and/or a change in an angular orientation of gauge 312, a spinal implant or an anatomy measured by and/or connected with gauge 312, similar to that described herein.

For example, gauge 312 is disposable in a first orientation, such as, for example, a calibration orientation. Lock 370 is disengaged from scale 342 such that scale 342 is disposed in a non-locking orientation and scale 342 rotates about a resting and/or equilibrium orientation. Gauge 312 is manipulated to a calibration orientation such that surface 320 engages a spinal implant and/or anatomy and is disposed in a selected orientation with the spinal implant and/or anatomy. Scale 342 rotates from rest and/or equilibrium and a restoring force due to gravity is subjected to scale 342. As such, scale 342 is rotated to a calibration orientation, as described herein. Lock 370 engages scale 342 in a locked orientation to fix scale 342 relative to arm 314 in the calibration orientation.

Gauge 312 is disposable in a second orientation, such as, for example, an angle measurement orientation, relative to the calibration orientation. Scale 342 is disposed in the locked orientation with arm 314, and pointer 344 rotates about a resting and/or equilibrium orientation. Gauge 312 is manipulated such that surface 320 engages the same or a different spinal implant and/or the same or a different portion of the anatomy for disposal in a selected orientation relative to the position of gauge 312 in the calibration orientation. Pointer 344 rotates from rest and/or equilibrium and a restoring force due to gravity is subjected to pointer 344. As such, pointer 344 is rotated to an angle measurement orientation relative to the calibration orientation of scale 342. Pointer 344 rotates relative to scale 342 such that pointer 344 is aligned with indicia 364 to represent and display an angular measurement of the angular difference between the engagement of surface 320 with the spinal implant and/or anatomy of gauge 312 in the calibration orientation and the engagement of surface 320 with the spinal implant and/or anatomy of gauge 312 in the angular measurement orientation, as described herein.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A surgical instrument comprising:
an arm;
a first member being movable relative to the arm and gravity responsive to define a first orientation of the arm;
a lock engageable with the first member; and
a second member movable relative to the arm and gravity responsive to measure an angle of a second orientation of the arm relative to the first orientation.

2. A surgical instrument as recited in claim 1, wherein the arm includes an engagement surface connectable with a spinal implant or an anatomy.

3. A surgical instrument as recited in claim 1, wherein the arm includes a bifurcated portion that defines a cavity configured for disposal of the members.

4. A surgical instrument as recited in claim 1, wherein the arm defines a longitudinal axis and the members are connected with the arm along a transverse axis.

5. A surgical instrument as recited in claim 1, wherein at least one of the members is rotatable relative to the arm.

6. A surgical instrument as recited in claim 1, wherein at least one of the members comprises a pendulum that is pivotable relative to the arm.

7. A surgical instrument as recited in claim 1, wherein the first member comprises a pendulum that is pivotable relative to the arm and the second member comprises a pendulum that is pivotable relative to the arm and the first member in a locked orientation.

8. A surgical instrument as recited in claim 1, further comprising indicia representing the angle.

9. A surgical instrument as recited in claim 1, further comprising indicia representing the angle, the indicia including graduated markings disposed with the first member and a pointer disposed with the second member.

10. A surgical instrument as recited in claim 1, wherein the first orientation includes a zero angle reference.

11. A surgical instrument as recited in claim 1, wherein the lock includes a push button engageable with the first member in the first orientation at a zero angle calibration.

12. A surgical instrument as recited in claim 1, wherein the first member comprises a scale that is pivotable relative to the arm.

13. A surgical instrument as recited in claim 1, wherein the first member comprises a scale that is pivotable relative to the arm and the second member comprises a ball that moves relative to the scale in a locked orientation.

14. A surgical instrument as recited in claim 13, wherein the scale includes a track configured for disposal of the ball.

15. A surgical instrument comprising:
an arm defining a longitudinal axis and including an engagement surface connectable with a spinal implant or an anatomy, the arm being movable between a first orientation and a second orientation;
a first pendulum connected with the arm along a transverse axis and being pivotable relative to the arm to an angle of the first orientation;
a lock engageable with the first pendulum to calibrate the first pendulum at the angle of the first orientation; and
a second pendulum connected with the arm along the transverse axis and being pivotable relative to the arm to measure an angle of the second orientation of the arm relative to the first orientation.

16. A surgical instrument as recited in claim 15, wherein the arm includes a bifurcated portion that defines a cavity configured for disposal of the pendulums.

17. A surgical instrument as recited in claim 15, further comprising indicia representing the angle, the indicia including graduated markings disposed with the first pendulum and a pointer disposed with the second pendulum.

18. A surgical instrument as recited in claim 15, wherein the engagement surface includes spaced apart arms that define an implant cavity.

19. A surgical instrument comprising:
an arm defining a longitudinal axis and including an engagement surface connectable with a spinal implant or an anatomy, the arm being movable between a first orientation and a second orientation;
a scale being rotatable relative to the arm and gravity responsive to an angle of the first orientation, the scale including a track;
a lock engageable with the scale to calibrate the scale at the angle of the first orientation; and
a ball movable along the track and gravity responsive to measure an angle of a second orientation of the arm relative to the first orientation.

20. A surgical instrument as recited in claim 19, further comprising indicia representing the angle, the indicia including graduated markings disposed with the first scale and the ball.
